(19) United States
(12) Patent Application Publication
(10) Pub. No.: US 2006/0293570 A1
(43) Pub. Date: Dec. 28, 2006

(54) Methods and apparatus for remotely enabling personal independence

(76) Inventors: John E. Croghan, Kenilworth, IL (US); Philip H. Sheridan JR., Winnetka, IL (US); Jonathan Weatherly, Chicago, IL (US); David Hughes, Highland Park, IL (US); Mark Skolnik, Highland Park, IL (US)

Correspondence Address: James F. Goedken;Bell, Boyd & Lloyd LLC, P.O. Box 1135, Chicago, IL, 60690-1135, US

(21) Appl. No.: 11/166,568

(22) Filed: Jun. 24, 2005

(51) Int. Cl. A61B 5/00

(52) U.S. Cl. 600/300

(57) ABSTRACT

Methods and apparatus for in-residence care are disclosed. The methods and apparatus enable a remote care provider to configure a home with a home controller and a plurality of medical and non-medical sensors based on the results of a periodic assessment survey. The remote care provider may also download software to the home controller to allow a new sensor to be connected to the home controller. The remote care provider then monitors data from the sensors electronically and provides services based on the assessment survey. Periodically and/or based on alerts generated in response to the monitored data, the remote care provider conducts virtual visits to the home using an audio/video telecommunications system. A home care provider or other professional may also conduct an actual visit to the home, and data associated with the actual visits may be recorded by the remote care provider.

### TECHNICAL FIELD

The present application relates in general to a care and management system, and, in particular, to methods and apparatus for remotely enabling personal independence at a home or other residence.

### BACKGROUND

Many seniors (and other individuals) and their adult children (or other loved ones) eventually face the decision of whether to place the senior in an adult care facility. This decision is often brought about by a medical condition. However, most seniors prefer to remain in their own home where they feel comfortable, independent, and secure. Staying at home is often less expensive than moving into an adult care facility especially in view of the rising cost of adult care facilities.

Many separate non-integrated services are available that are designed to prolong the amount of time a senior can comfortably and safely remain in their own home. For example, nurses and/or aides may be hired to visit the senior on a periodic basis for scheduled assistance such as medical checkups and delivery of medication. Similarly, many separate non-integrated products are also designed to prolong the amount of time a senior can comfortably and safely remain in their own home. For example, a blood glucose monitor may transmit data to a central monitoring facility. Likewise, many other non-integrated products and services are available to people to provide better lifestyles and living. For example, numerous food delivery services are also available to ensure that people at home are getting food that is enjoyable, varied, and healthy. Numerous other services such as home cleaning and maintenance services are also available.

However, these existing products and services do not provide a complete care system for the person, in part, because they are not integrated with each other. The complete care of most elderly people includes providing for medical and non-medical needs in a prescriptive manner based on a geriatrician's review of the individual's overall needs (e.g., make sure the person complies with his/her medications regime consistently, has someone come in the home to do light housework, and have a family member or caregiver visit daily to check on meals, and has someone to make sure the person's bills are being paid). Taking care of traditionally non-medical needs for the senior (e.g., facilitating attendance at social events, handling bills, facilitating home maintenance, providing different types of food, etc.) simplifies the home environment for the senior and greatly reduces stress, which often leads to better mental and even physical health for the person.

If a seniors are not worried about potentially stressful things (e.g., whether the bills are getting paid on time, whether their home is clean, and whether they have taken all of their medication), they are typically happier and healthier. Similarly, if seniors are getting regular and meaningful human interaction, they are typically happier and healthier. For example, if an adult child calling a parent knew what that parent did that day (e.g., where they went, what they ate), the adult child could ask the parent meaningful questions about their day (e.g., How was your dinner with your bridge group?). Care systems that only take care of part of the person (not in accordance with the geriatrician view) suffer from many drawbacks.

First, the selection and installation of these products and services are not performed in an integrated manner. For example, if the senior requires two different medical devices to transmit medical data via the telephone line, it is up to the user or the clever design of the individual medical devices to make sure one device is not trying to communicate via the telephone line at the same time another device is communicating via the telephone line.

Second, because the selected products and services are not under a single umbrella of control, the data generated by these products and services is not available in a single comprehensive report readily viewable by caretakers of the person, such as a son or daughter. As a result, alerts based on a combination of data from different sensors cannot be generated. For example, a sudden increase in blood sugar reported by a glucose monitor may be easily explained by a food item reported by a visiting aide or delivered by a service that is integrated within a total care system, if these two data points were reported together. However, no such systems currently exist for home care or living situations.

Third, the selection of the needed products and/or services is typically a one time event and/or is reactive in nature. For example, it may not be discovered that the senior is mishandling his/her bills until after a certain amount of financial damage is done. In response, the senior may be provided with a bill paying service. Again, data associated with this bill paying service is not integrated with other data coming from the other home care products and services. Although there are multiple assessment tools for Seniors, they typically: (i) only cover one or a small number of issues (e.g., dementia or mobility, not both) (ii) focus exclusively on medical or health-related issues, often ignoring home office and home care issues (e.g., bill payment, claims management, and repairs) and social issues (e.g., interaction time with family and others) that can be just as important in assessing overall needs in the home, and (iii) only provide scores, not useful recommendations for action (e.g., dementia score of 19 out of possible 30 and mobility score of 14 out of possible 20; no recommendation of how these and other scores relate to a specific set of recommendations).

Fourth, very valuable information is typically gathered from a periodic face-to-face visit with the senior. For example, the “How are you feeling?” question typically asked by doctors and nurses often reveals valuable non-numerical information. However, visiting the senior is also one of the most costly services to provide. Accordingly, there is a need for a complete in-residence care system.

### SUMMARY

Methods and apparatus for in-residence care that solve these problems are disclosed. An in-person physician or nurse, or a remote care provider administers a needs assessment survey to determine what products and/or services the particular senior (or other person such as a disabled child) would most benefit from. The needs assessment survey is designed to deliver a “prescription” of products and services for the senior, not just a score or a list of products for a home. This proprietary assessment tool uses scores across a range of issues to deliver a specific “managed daily activity” recommendation, specific to the unique needs of the Senior. For example, the assessment tool reviews specific medical and health issues (e.g., specific diseases or pain), mobility, activities of daily living, and home office (e.g., bill payment) and social activities, among others. Based on the use of this tool, it may recommend to Senior #1 that they use a video RN visit once per week, utilize a “blister pack” for medication dispensing, use companion services for light housework, and that they install activity monitoring devices in the bathroom and kitchen. The same tool may recommend to Senior #2 that they use a video RN visit daily, use an advanced electronic device for medication dispensing, use a visiting nurse each morning to help with transfer, and that they have activity sensors in the bathroom, kitchen, bed, and front door and back door, and that they utilize an on-line bill management system through a designated family member or power of attorney. This assessment tool delivers a different recommendation for Senior #1 and Senior #2 because it is based on the different needs of each senior.

Based on the initial needs assessment survey, the remote care provider configures the home with a home controller and a plurality of medical and non-medical sensors in one integrated package. For example, the senior may need basic well being or activity monitoring, safety monitoring, and medication reminders. In this example, a plurality of motion detectors may be associated with different rooms of the home, a wireless weight scale may be installed in the bathroom, detection devices may be installed on oven doors and exterior doors, and reminders may be scheduled for medication. All of these different types of sensors are connected (wired or wirelessly) to a remotely upgradeable home controller.

The remote care provider may also provide one or more non-monitoring services (i.e., support services) based on the results of the needs assessment survey. Support services are activities performed electronically, mechanically or by a person that makes up for, adds on to or replaces activities that would otherwise be performed by a person living at home or in the course of daily living. Support services help reduce stress for the senior. For example, the remote care provider may provide and/or contract for bill handling services, mail handling services, legal document generation services, tax return preparation services, claims management services, transportation coordination services (to doctors, senior centers and other places of interest for the person), food preparation services, shopping services, laundry services, housekeeping services, home maintenance services, etc.

Because seniors typically have changing needs as they become older (e.g., new medical conditions), the remote care provider periodically re-administers or updates the needs assessment survey. If a new sensor is required, and that new sensor is not already compatible with the home controller, the remote care provider may download suitable software to the home controller to allow the new sensor to be connected to the home controller. If the sensor is simple enough for the senior to “install” (e.g., a wireless scale simply needs to be placed on the floor), the remote care provider preferably downloads the software before the new sensor arrives at the home. Preferably, this updating of the system is proactive in nature (e.g., we need to start measuring your weight because you are at the age where weight loss is an indicator of condition X).

The home controller receives data from the sensors and sends the data to a central database preferably at the remote care provider's facilities. For example, the home controller may transmit the data to the central database via the Internet or a telephone line. Because the system is integrated into a single package or system, software at the remote care provider may monitor the data for a plurality of different predetermined alert conditions. For example, the software may be programmed to generate an alert if the oven door has been open for longer than five minutes or the senior has been in the bathroom for longer than one hour. Also, based on specific input from a physician selected by the senior, the system may trigger alerts if specific medical measurements occur (e.g., blood pressure below 105 or number of sleeping hours exceeding 12).

In addition, the data may be used to generate customized reports showing the daily living activities of the senior. For example, the remote care provider may send one report with one set of information to each adult child every day, week or other suitable period via e-mail (e.g., Mom got out of bed and her usual time, went to bridge club, made her own lunch, took her medicine, had Chinese food delivered for dinner, and went to bed at her normal time) and another report with different information to the family doctor once a month via fax (e.g., weight, blood pressures, etc.). In this manner, adult children can rest assured that their parent is living normally and can use the information from the reports to initiate meaningful dialog with the parent. On the other hand, the physician can use his/her report to monitor the medical needs of the senior. The type, frequency, and delivery method for each report is preferably selectable by the each report recipient (e.g., via a suitable web page or other suitable method).

In response to a preprogrammed schedule and/or in response to an alert, the home care provider may electronically contact the senior with preprogrammed messages. For example, if the sensor data indicates that the senior has not taken his/her medication on schedule, a computer at the home care provider facility may dial the senior's telephone and play a prerecorded message reminding the senior to take their medication. These reminders may also be audio/video or other suitable messages.

Periodically and/or based on alerts generated in response to the monitored data, the remote care provider conducts virtual visits to the home. These virtual visits use an audio/video telecommunications systems installed in the home and at the home care provider's facility. For example, the remote care provider may schedule a virtual visit where the senior's weight and blood pressure are to be discussed with the senior. Accordingly, if the senior's weight and blood pressure have not been recorded within some predetermined period of time in the past, the system may send an automatic reminder to the senior to take these measurements in advance of the virtual visit so that time during the virtual visit is not spent gathering this data. The nurse may then contact the senior via the audio/video system, asks the senior certain questions, and record data and remarks associated with the virtual visit. The remote care provider also stores this data and these remarks in the central database at the remote care provider's facilities.

A home care provider or other professional may also conduct an actual visit to the home, and the remote care provider may store data associated with the actual visits. For example, a visiting nurse may use a wireless tablet computer to record data and remarks. The wireless tablet computer connects to the home controller, which in turn transmits the data to the central database at the remote care provider's facilities. Alternatively, an automated phone based system may prompt the actual visitor for answers to a short list of questions specific to that senior. The prompts may be in any language (based on the needs of the home care visitor), and the responses may be touch tone responses (e.g., press 1 for yes) or verbal responses recorded by the remote care provider.

Other objects, features and advantages of the system will be apparent from the following detailed disclosure, taken in conjunction with the accompanying sheets of drawings, wherein like numerals refer to like parts, elements, components, steps and processes.

### BRIEF DESCRIPTION OF THE DRAWINGS

FIG. 1 is a block diagram of an example communications network connecting a plurality of devices according to one embodiment of the methods and apparatus of the present system.

FIG. 2 is a block diagram of an example electronic configuration of one embodiment of the home controller of FIG. 1.

FIG. 3 is a block diagram of an example electronic configuration of one embodiment of the database server of FIG. 1.

FIG. 4 is a block diagram of an example electronic configuration of one embodiment of the monitoring station of FIG. 1.

FIG. 5 is a flowchart of an example process for providing in-residence care according to one embodiment of the methods and apparatus of the present system.

### DETAILED DESCRIPTION OF EXEMPLARY EMBODIMENTS

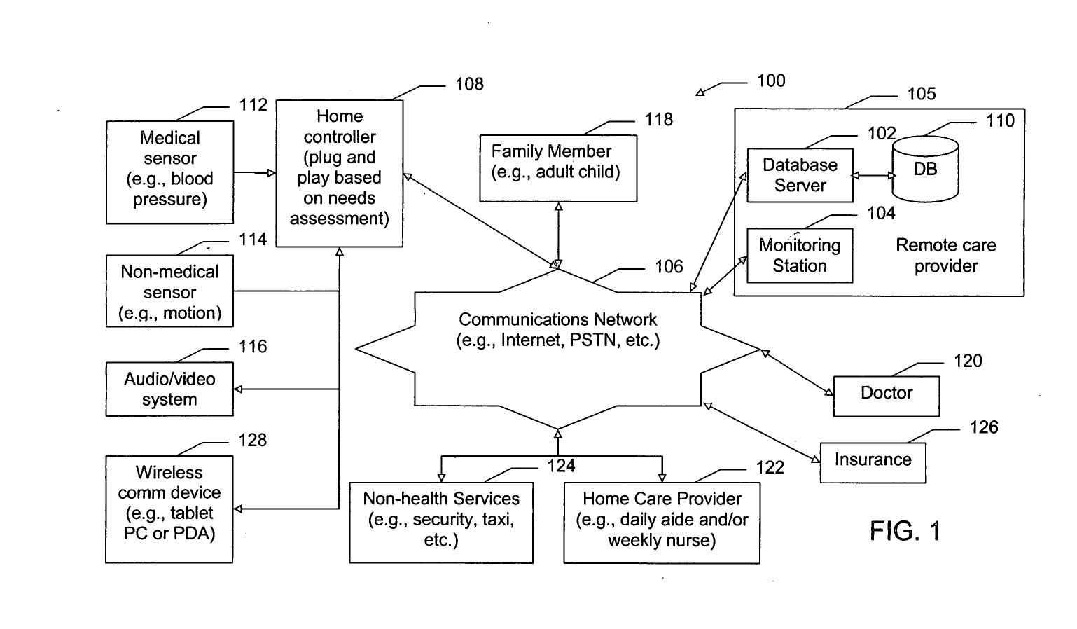

The present system (i.e., methods and apparatus) is most readily realized in a network communications system. A block diagram of an exemplary network communications system 100 is illustrated in FIG. 1. The illustrated system 100 includes one or more database servers 102 and one or more monitoring stations 104 of a remote care provider 105 connected via a network 106 to a plurality of home controllers 108. The network 106 may be any type of suitable local or wide area network, such as a secure Ethernet network and/or a fiber optic network. It will be appreciated that any of the devices described herein may be directly connected to each other and/or over a network through a conventional phone line, a digital signal line (DSL), a T-1 line, a coaxial cable, a fiber optic cable, and/or any other suitable connection.

The databases 110 may be part of the database servers 102 and/or connected via the network 106. One database server 102 may interact with a large number of home controllers 108. Accordingly, each database server 102 is typically a high end computer with a large storage capacity, one or more fast microprocessors, and one or more high speed network connections. Conversely, relative to a typical database server 102, each home controller 108 typically includes less storage capacity and computing power.

The database server 102 stores a plurality of files and/or programs in one or more databases 110 for use by the monitoring stations 104 and/or the home controllers 108. For example, the database server 102 may store data from one or more medical sensors 112 and/or one or more non-medical sensors 114. As described in more detail below, the database server 102 may use this sensor data to generate alerts. For example, if an oven door is left open for more than a predetermined period of time, the database server 102 may generate an alert indicating that the oven door has been left open.

The monitoring station 104 communicates with the database server 102 and the home controllers 108. For example, the oven door alert may be sent to the monitoring station 104. In response to an alert and/or at some periodic time, a person at the monitoring station 104 may then perform a virtual visit to the associated home via an audio/video telecommunications system 116. For example, the person at the monitoring station 104 may contact the person in the residence to follow up on why the oven door is open and/or to perform a mental health status check. In addition, the person at the monitoring station 104 may query the database 110 to view data associated with the person in the residence.

Preferably, a plurality of other devices are also connected to the network 106 and in communication with the database server 102 and/or the home controllers 108. For example, the database server 102 may send alerts and/or periodic reports to one or more family member computers 118 via the network 106. Similarly, the database server 102 may send alerts and/or periodic reports to one or more doctor computers 120, one or more home care provider computers 122, and/or one or more other services providers 124. Different report recipients may receive the same and/or different reports. For example, family members may receive general status updates, doctors may receive medical readings, and home care providers may receive data associated with aide visits. Preferably, the report recipient selects the period and type of the report via a web page on the Internet. Any report may include trended data (e.g., daily weight or glucose level). In addition, the database server 102 may send an insurance claim to a doctor computer 120 and/or an insurance computer 126 (e.g., Medicare, a private insurance company, or a government insurance program).

To receive certain data associated with aide (or other professional) visits, the home controller 108 preferably is capable of receiving data from a wireless communications device 128, such as a tablet computer or a personal digital assistant (PDA). For example, a visiting nurse may take medical readings and/or ask the person in the home certain predetermined questions. The visiting professional may then enter the results of the medical readings and/or responses to the predetermined questions into the wireless communications device 128. The wireless communications device 128 sends this data to the database server 102 via the home controller 108. In addition, the person visiting the residence may query the database 110 to view data associated with the person they are visiting.

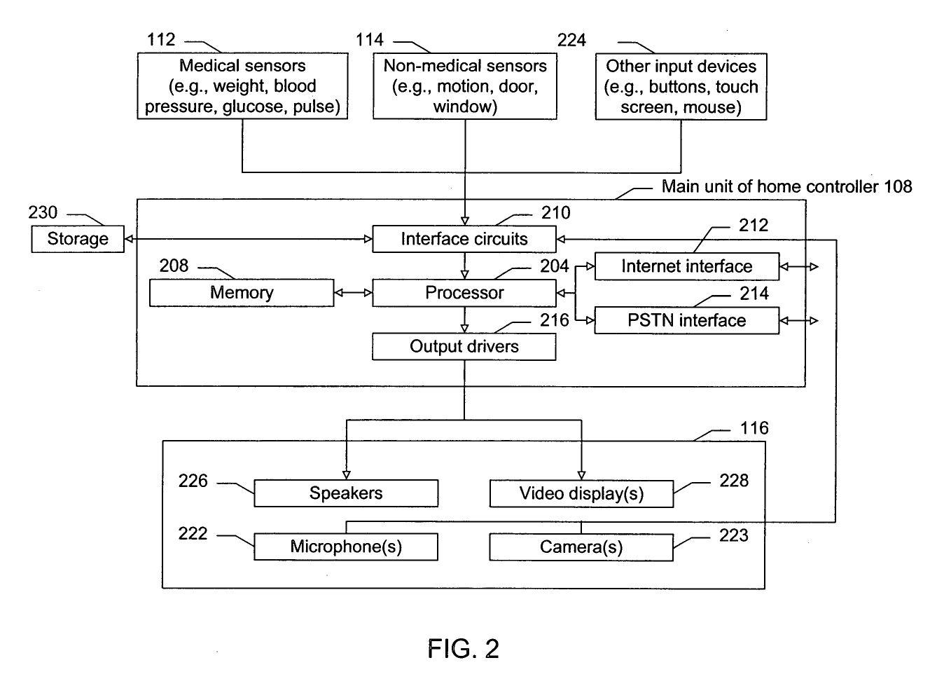

A more detailed block diagram of a home controller 108 is illustrated in FIG. 2. The home controller 108 may include a personal computer (PC) and/or any other suitable computing device. The home controller 108 preferably includes a main unit 108 which preferably includes one or more processors 204 electrically coupled by an address/data bus to one or more memory devices 208, one or more interface circuits 210, 212, and 214, and one or more output drivers 216. The processor 204 may be any suitable processor, such as a microprocessor, a microcontroller-based platform, a suitable integrated circuit or one or more application-specific integrated circuits (ASIC's).

The memory 208 preferably includes volatile memory and/or non-volatile memory. In an embodiment, the memory device includes random access memory (RAM), read only memory (ROM), flash memory, and/or electrically erasable programmable read only memory (EEPROM). Any suitable memory may be used. Preferably, the memory 208 stores a software program that interacts with the other devices in the system 100 as described below. This program may be executed by the processor 204 in any suitable manner. However, some of the steps described below in connection with the methods may be performed manually and/or without the use of the home controller 108. In one embodiment, part or all of the program code can be stored in a detachable or removable memory device, including, but not limited to, a suitable cartridge, disk or CD ROM. The memory 208 may also store digital data indicative of documents, files, programs, web pages, videos, still images, etc. retrieved from another computing device and/or loaded via an input device.

The interface circuits 210, 212, and/or 214 may be implemented using any suitable interface standard(s), such as an Ethernet interface, a wireless interface (e.g., IEEE 802.11) a Universal Serial Bus (USB) interface, and/or a public switched telephone network (PSTN) interface. Preferably, one or more medical sensors 112 and one or more non-medical sensors 114 are connected to the main unit 108 via one or more interface circuits 210. The sensors 112 and/or 114 may be wired or wireless. Medical sensors are sensors that measure physiological parameters, either normal or abnormal, such as weight, temperature, blood glucose level, blood pressure, heart rate, blood oxygenation and others. Examples of medical sensors include scales, blood pressure sensors, blood sugar meters, heart rate monitors, etc. Examples of non-medical sensors include motion detectors, door position sensors, window position sensors, heat detectors, smoke detectors, panic buttons, etc.

One or more microphones 222 and cameras 223 may also be connected to the main unit 108 via the interface circuit(s) 210. As described in more detail below, the microphones 222 and cameras 223 form part of a teleconferencing system used to perform virtual visits to the monitored residence. In one embodiment, the camera may be configured to selectively acquire still or moving (e.g., video) images and may be configured to acquire the images in either an analog, digital or other suitable format. In addition, other input devices 224 may be connected to the interface circuits 210 for entering data and commands into the main unit 108. For example, the input device may be a keyboard, mouse, touch screen, track pad, track ball, isopoint, and/or a voice recognition system may be used.

One or more speakers 226, displays 228, and/or other output devices may also be connected to the main unit 108 via the interface circuit(s) 210. The display 228 may be a cathode ray tube (CRTs), liquid crystal displays (LCDs), or any other type of suitable display. In one embodiment, the display 228 includes a touch-screen with an associated touch-screen controller. The display 228 generates visual displays of data generated during operation of the main unit 108 and/or video for the teleconferencing system. For example, the display 226 may be used to display web pages received from the database server 102 and/or images of a person's face from the monitoring station 104.

One or more storage devices 230 may also be connected to the main unit 108 via the interface circuit(s) 210. For example, a hard drive, CD drive, DVD drive, and/or other suitable storage devices may be connected to the main unit 108. The storage devices 230 may store any type of data used by the home controller 108.

Users of the system 100 may be required to register with the home controller 108 and/or the database server 102. In such an instance, each user may choose a user identifier (e.g., e-mail address) and a password which may be required for the activation of services. The user identifier and password may be passed across the network 106 using encryption built into the user's browser. Alternatively, the user identifier and/or password may be assigned by the database server 102.

Certain users may connect to the database server 102 to access data and view or generate reports. Access to the database server 102 and reports can be controlled by appropriate security software or security measures. An individual member's access can be defined in the system and limited to certain data, information and reports. Access to non-authorized data, information, and reports is preferably prohibited.

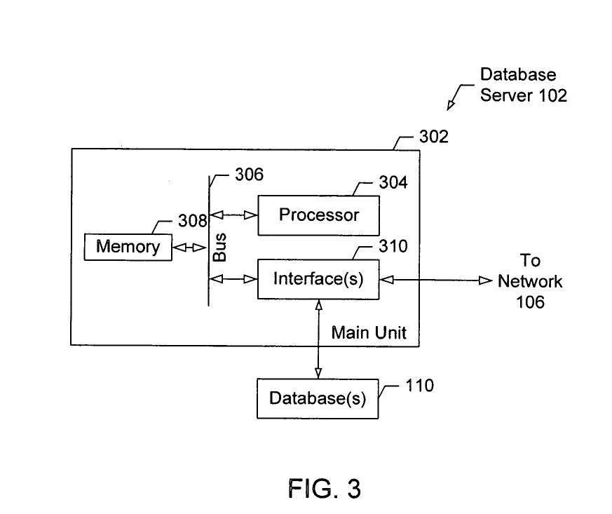

A more detailed block diagram of a database server 102 is illustrated in FIG. 3. A main unit 302 in the database server 102 preferably includes a processor 304 electrically coupled by an address/data bus 306 to a memory device 308 and a network interface circuit 310. The database server 102 may exchange data with other devices via a connection to the network 106. The network interface circuit 310 may be implemented using any suitable data transceiver, such as an Ethernet transceiver.

The processor 304 may be any type of suitable processor such as a microprocessor, a microcontroller-based platform, a suitable integrated circuit or one or more application-specific integrated circuits (ASIC's). The memory 308 preferably includes volatile memory and/or non-volatile memory. In an embodiment, the memory device includes random access memory (RAM), read only memory (ROM), flash memory, and/or electrically erasable programmable read only memory (EEPROM). Any suitable memory may be used.

Preferably, the memory 308 stores a software program that interacts with the other devices in the system 100 as described below. This program may be executed by the processor 304 in any suitable manner. However, some of the steps described below in connection with the methods may be performed manually and/or without the use of the database server 102. In one embodiment, part or all of the program code can be stored in a detachable or removable memory device, including, but not limited to, a suitable cartridge, disk or CD ROM. The memory device 308 and/or a separate database 110 may also store files, programs, web pages, etc. for use by home controllers 108, the monitoring station 104, and/or other devices connected to the network 106.

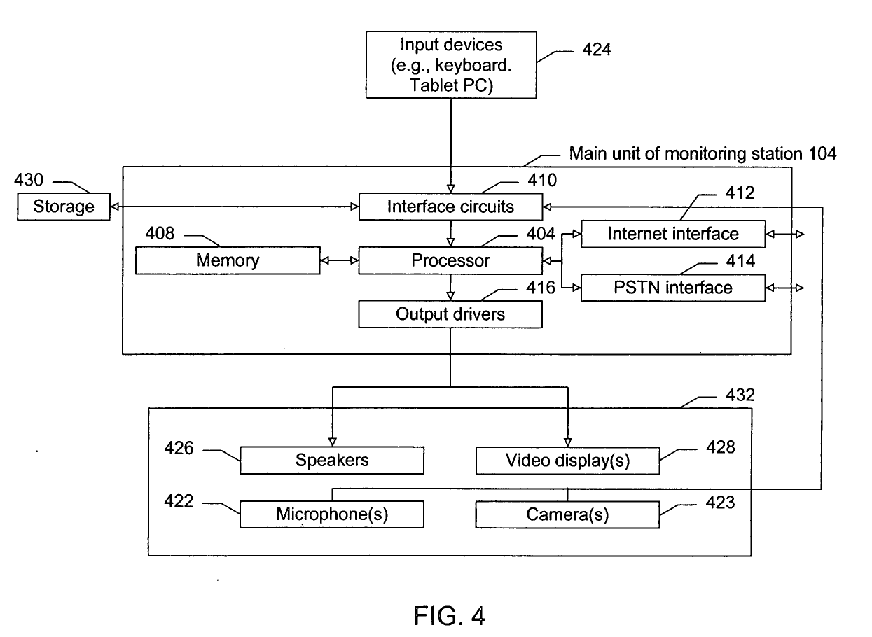

A more detailed block diagram of a monitoring station 104 is illustrated in FIG. 4. The monitoring station 104 may include a personal computer (PC) and/or any other suitable computing device. The monitoring station 104 preferably includes a main unit 104 which preferably includes one or more processors 404 electrically coupled by an address/data bus to one or more memory devices 408, one or more interface circuits 410, 412, and 414, and one or more output drivers 416. The processor 404 may be any suitable processor, such as a microprocessor, a microcontroller-based platform, a suitable integrated circuit or one or more application-specific integrated circuits (ASIC's).

The memory 408 preferably includes volatile memory and/or non-volatile memory. In an embodiment, the memory device includes random access memory (RAM), read only memory (ROM), flash memory, and/or electrically erasable programmable read only memory (EEPROM). Any suitable memory may be used. Preferably, the memory 408 stores a software program that interacts with the other devices in the system 100 as described below. This program may be executed by the processor 404 in any suitable manner. However, some of the steps described below in connection with the methods may be performed manually and/or without the use of the home controller 104. In one embodiment, part or all of the program code can be stored in a detachable or removable memory device, including, but not limited to, a suitable cartridge, disk or CD ROM. The memory 408 may also store digital data indicative of documents, files, programs, web pages, etc. retrieved from another computing device and/or loaded via an input device.

The interface circuits 410, 412, and/or 414 may be implemented using any suitable interface standard(s), such as an Ethernet interface, a wireless interface (e.g., IEEE 802.11) a Universal Serial Bus (USB) interface, and/or a public switched telephone network (PSTN) interface.

One or more microphones 422 and cameras 423 may also be connected to the main unit 104 via the interface circuit(s) 410. As described in more detail below, the microphones 422 and cameras 423 form part of a audio/video telecommunications system 432 used to perform virtual visits to the monitored residence. In one embodiment, the camera may be configured to selectively acquire still or moving (e.g., video) images and may be configured to acquire the images in either an analog, digital or other suitable format. In addition, other input devices 424 may be connected to the interface circuits 410 for entering data and commands into the main unit 104. For example, the input device may be a keyboard, mouse, touch screen, track pad, track ball, isopoint, and/or a voice recognition system may be used.

One or more speakers 426, displays 428, and/or other output devices may also be connected to the main unit 104 via the interface circuit(s) 410. The display 428 may be a cathode ray tube (CRTs), liquid crystal displays (LCDs), or any other type of suitable display. In one embodiment, the display 428 includes a touch-screen with an associated touch-screen controller. The display 428 generates visual displays of data generated during operation of the main unit 104 and/or video for the teleconferencing system. For example, the display 426 may be used to display web pages received from the database server 102 and/or images of a person's face from a home controller 108.

One or more storage devices 430 may also be connected to the main unit 104 via the interface circuit(s) 410. For example, a hard drive, CD drive, DVD drive, and/or other suitable storage devices may be connected to the main unit 104. The storage devices 430 may store any type of data used by the monitoring station 104.

Users of the system 100 may be required to register with the monitoring station 104. In such an instance, each user may choose a user identifier (e.g., e-mail address) and a password which may be required for the activation of services. The user identifier and password may be passed across the network 106 using encryption built into the user's browser. Alternatively, the user identifier and/or password may be assigned by the database server 102.

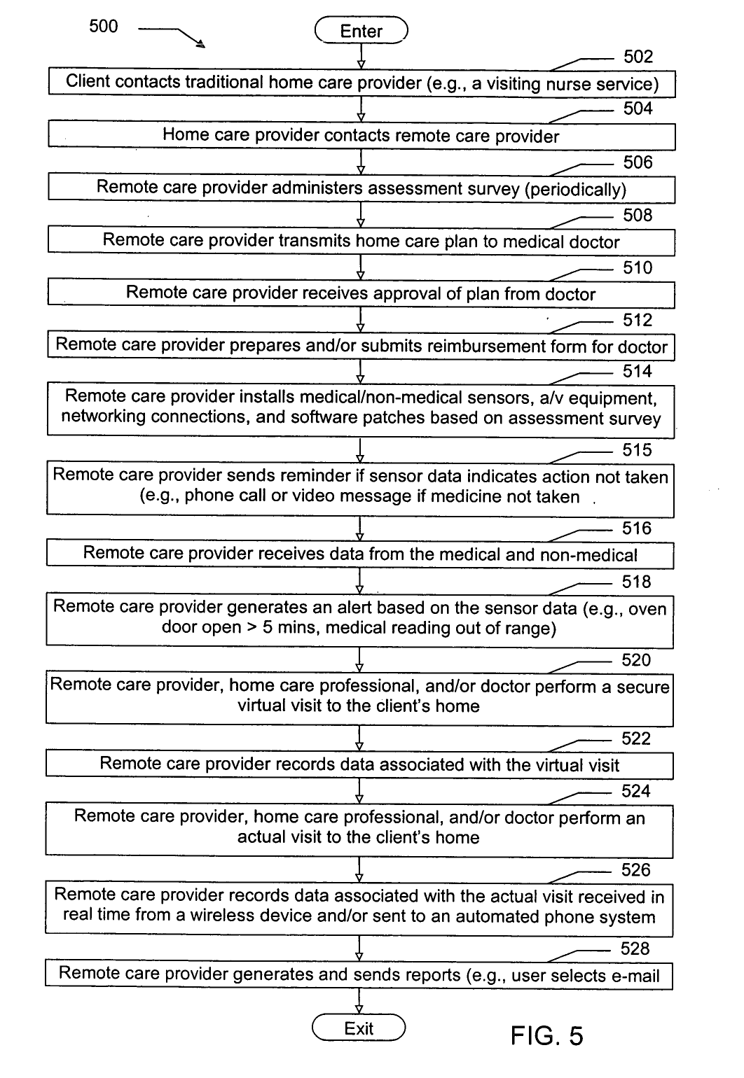

A flowchart of an example process 500 for providing in-residence care is illustrated in FIG. 5. Preferably, the process 500 is embodied in one or more software programs which is stored in one or more memories and executed by one or more processors. Although the process 500 is described with reference to the flowchart illustrated in FIG. 5, it will be appreciated that many other methods of performing the acts associated with process 500 may be used. For example, the order of many of the blocks may be changed, and many of the blocks described may be optional.

Generally, the process 500 enables a remote care provider 105 to configure a home with a home controller 108, one or more medical sensors 112, and one or more non-medical sensors 114 based on the results of an assessment survey and the approval of a medical doctor. The remote care provider 105 then monitors data from the sensors 112 and 114. Periodically and/or based on alerts generated in response to the monitored data, the remote care provider 105 conducts virtual visits to the home using an audio/video telecommunications system. A home care provider or other professional may also conduct an actual visit to the home, and data associated with the actual visits may be recorded by the remote care provider 105.

The process 500 begins when a client contacts a home care provider 122 as indicated by block 502. For example, an elderly or disabled person living in a home may contact a nursing company to contract a weekly visit. The home care provider 122 then contacts the remote care provider 105 as indicated by block 504. For example, the remote care provider 105 may have a contractual relationship with the home care provider 122.

The remote care provider 105 then administers a needs assessment survey as indicated by block 506. A needs assessment survey is a survey that determines a client's medical and non-medical needs. The assessment collects information about a person's medical, physical, psychological, familial, social and environmental conditions as well as ability to perform the full range of activities of daily living, including but not limited to ADL's, IADL's and EADL's. The assessment is both evaluative and prescriptive, with answers to one of more questions providing an evaluation of the abilities or inabilities of the person to function independently at their residence and prescribing one or more monitoring or support functions, services, or medical or non-medical modifications that will assist that person in functioning at their residence. The assessment survey preferably includes questions related to the client's health needs (e.g., a mental health examination), home office needs (e.g., bill handling, mail handling, health claims administration, tax handling, legal administration, etc.), activities of daily living needs (e.g., meals, medication, dressing, bathing, toileting, laundry, groceries, eating, house cleaning, home repair, lawn care, snow removal, etc.), and/or social needs (e.g., events, transportation, etc.). The residence associated with the survey may be self owned (e.g., a home, a condominium, or an apartment) or care based (e.g., an assisted living facility or an independent living facility).

Based on the completed assessment survey, the remote care provider 105 develops a home care plan and transmits the plan to a medical doctor for approval as indicated by block 508. If the home care plan is approved, the remote care provider 105 receives an approval from the medical doctor as indicated by block 510. If the home care plan is not approved, the remote care provider 105 and/or the doctor adjusts the home care plan. Once approved, the remote care provider 105 preferably prepares and/or submits the appropriate reimbursement form to an insurance company for the doctor as indicated by block 512.

Based on the needs assessment survey, the remote care provider 105 installs the needed medical sensors, non-medical sensors, audio/video equipment, and networking connections in the client's home as indicated by block 514. For example, the remote care provider 105 may install motion detectors, door position sensors, window position sensors, scales, blood pressure sensors, blood sugar meters, heart rate monitors, etc. The sensors may be connected to the home controller 108 via a direct connection and/or via a wireless connection.

Also based on the needs assessment survey, the remote care provider 105 sets up the needed non-monitoring services. Non-monitoring services are services that are not related to the medical sensors 112 and non-medical sensors 114 installed in the residence. For example, the remote care provider may provide and/or contract for bill handling services, mail handling services, legal document generation services, tax return preparation services, claims management services, transportation coordination services, food preparation services, shopping services, laundry services, housekeeping services, home maintenance services, etc.

Periodically, the needs assessment survey is re-administered. If a new sensor is required, and that new sensor is not already compatible with the home controller, the remote care provider may download software patches to the home controller to allow the new sensor to be connected to the home controller. If the sensor is simple enough for the senior to “install” (e.g., a wireless scale simply needs to be placed on the floor), then the software download is preferably performed in advance of the new sensor being received at the home.

The remote care provider 105 then receives data from the sensors as indicated by block 516. Preferably, the home controller 108 automatically transmits the data to the database server 102 via the network 106. Software in the database server 102 (or some other suitable computing device) examines the sensor data according to a plurality of predetermined rules to determine if an alert should be generated as indicated by block 518. For example, if an oven door sensor indicates the oven door has been open longer than some predetermined time (e.g., 5 minutes), then the database server 102 may generate an alert.

In response to an alert and/or at some periodic time, a person at the monitoring station 104 (or some other person) may then perform a virtual visit to the associated home via an audio/video telecommunications system 116 as indicated by block 520. For example, the person at the monitoring station 104 may contact the person to follow up on why the oven door is open and/or to perform a mental health status check. The database server 102 preferably records the data associated with the virtual visit as indicated by block 522. For example, the explanation for the oven door being open may be that the client was cleaning the oven.

Similarly, a person may perform an actual visit to the associated home as indicated by block 524. For example, a professional medical service provider (e.g., doctor, nurse, or aide), a companion service worker, and a professional non-medical service provider (e.g., accountant or lawyer) may visit the home and record data associated with the visit. The database server 102 preferably records the data associated with the actual visit as indicated by block 526. For example, a visiting nurse may take medical readings and/or ask the person in the home certain predetermined questions. The visiting professional may then enter the results of the medical readings and/or responses to the predetermined questions into a wireless communications device 128. The wireless communications device 128 sends this data to the database server 102 via the home controller 108.

Alternatively, an automated phone based system may prompt the visiting professional for answers to a short list of questions specific to that senior. The automated system may contact the visiting professional at the residence at a predetermined time, or the visiting professional may dial a predetermined phone number. The prompts may be in any language (based on the needs of the home care visitor). For example, the system may ask the visiting professional to enter the senior's weight. The responses may be touch tone responses or verbal responses. For example, the visiting professional could enter the senior's weight on the phone keypad or speak the answer in to the phone. The remote care provider 105 preferably records verbal responses digitally so the responses may be forwarded and/or retrieved to/by interested parties such as doctors and family members. The verbal response may also be recorded in an analog format and/or digitized after recording.

In summary, methods and apparatus for providing in-residence care have been provided. The foregoing description has been presented for the purposes of illustration and description. It is not intended to be exhaustive or to limit the invention to the exemplary embodiments disclosed. Many modifications and variations are possible in light of the above teachings. It is intended that the scope of the invention be limited not by this detailed description of examples, but rather by the claims appended hereto.

The invention is claimed as follows:

1. A method of remotely providing in-residence care, the method comprising:
causing a first needs assessment survey to be administered to a person;
causing a recommendation of at least one medical sensor, at least one non-medical sensor, and at least one non-monitoring service based on a result of the first needs assessment survey;
causing an installation of the at least one medical sensor and the at least one non-medical sensor in a residence of the person;
receiving an alert signal at a remote location, the alert signal being based on sensor data from the at least one medical sensor and the at least one non-medical sensor;
causing a second needs assessment survey to be administered to the person;
causing a selection of at least one new sensor based on a result of the second needs assessment survey;
causing a transmission of first audio/video signals to the residence; and
receiving second audio/video signals from the residence.

2. The method of claim 1, wherein the at least one non-monitoring service includes at least one medical service and at least one non-medical service.

3. The method of claim 1, wherein the at least one non-monitoring service includes at least one of bill handling, mail handling, legal document generation, tax return preparation, claims management, transportation coordination, food preparation, shopping, laundry, housekeeping, and home maintenance.

4. The method of claim 1, wherein the medical sensor and the non-medical sensor are wirelessly connected to a home controller.

5. The method of claim 1, wherein the first needs assessment survey includes the second needs assessment survey.

6. The method of claim 1, wherein causing the second needs assessment survey to be administered includes causing the second needs assessment survey to be administered at least thirty days after causing the first needs assessment survey to be administered.

7. The method of claim 1, including causing a transmission of a reminder to perform a periodic medical procedure to the residence.

8. The method of claim 7, wherein the medical procedure includes taking a medication.

9. The method of claim 7, wherein the medical procedure includes taking a medical test.

10. The method of claim 7, wherein transmitting the reminder includes transmitting third audio/video signals to the residence.

11. The method of claim 7, wherein transmitting the reminder includes playing a prerecorded audio message over a telephone line.

12. The method of claim 1, wherein transmitting the first audio/video signals to the residence is in response to the alert.

13. The method of claim 1, including:
causing a development of a home care plan based on results associated with the first needs assessment survey;
causing a transmission of the home care plan to a medical doctor;
receiving an approval for the home care plan from the medical doctor; and
causing a generation of a home care plan insurance claim.

14. The method of claim 13, including causing a transmission of a copy of the home care plan insurance claim to the medical doctor.

15. The method of claim 13, including submitting the home care plan insurance claim to an insurance entity for reimbursement directly to the medical doctor.

16. The method of claim 1, including receiving data from a visitor in the residence via a wireless communication device.

17. The method of claim 16, wherein the visitor is at least one of a professional medical service provider, a companion service worker, and a professional non-medical service provider.

18. The method of claim 1, including causing a generation of a report associated with a person living in the residence and causing an automatic transmission of the report to a family member of the person.

19. The method of claim 18, wherein a period and a type associated with the report are selectable by the family member via a global network.

20. The method of claim 1, including causing a generation of a report associated with the person living in the residence and automatically transmitting the report to a first home care provider and a second different home care provider.

21. The method of claim 1, including causing a generation of a report associated with the person living in the residence, the report including a remarks section, and automatically transmitting the report to a doctor associated with the person living in the residence.

22. The method of claim 1, including causing a generation of a report associated with the person living in the residence, the report including trended data, and automatically transmitting the report to a doctor associated with the person living in the residence.

23. The method of claim 1, wherein the first needs assessment survey is a periodic needs assessment survey and the recommendation of the at least one medical sensor, the at least one non-medical sensor, and the non-monitoring service is updated based on the periodic needs assessment survey.

24. The method of claim 1, wherein the first needs assessment survey includes a health component, an activities of daily living component, a home office component, and a social component.

25. The method of claim 24, wherein the health component includes a mental health examination.

26. The method of claim 24, wherein the activities of daily living component includes at least one of a meal portion, a medication portion, a dressing portion, a bathing portion, a laundry handling portion, a grocery handling portion, a house cleaning portion, a home repair portion, a lawn care portion, and a snow removal portion.

27. The method of claim 24, wherein the home office component includes at least one of a bill handling portion, a mail handling portion, a health claims administration portion, a tax handling portion, and a legal administration portion.

28. The method of claim 24, wherein the social component includes at least one of an events portion and a transportation portion.

29. The method of claim 1, wherein the sensor data includes data from at least one of a weight scale, a vitals device, a blood pressure device, a blood sugar device, and a heart rate device.

30. The method of claim 1, wherein the sensor data includes data from at least one of an alarm system, a motion detector, a door sensor, a window sensor, and a pressure sensor.

31. The method of claim 1, wherein the sensor data and the second audio/video signals are transmitted from the residence together in real time.

32. The method of claim 1, wherein a selected subset of sensor data is transmitted to a monitoring station for a virtual visit to the residence.

33. The method of claim 32, wherein the alert signal is further based on data collected from the virtual visit.

34. The method of claim 1, wherein the alert signal is based on a determination that a door has been open for a time that is greater than a predetermined threshold.

35. The method of claim 34, wherein the door is an appliance door.

36. The method of claim 1, wherein the alert signal is based on a determination that a person has been in a room for a time that is greater than a predetermined threshold.

37. The method of claim 1, wherein the alert signal is based on a determination that a person did not perform a medical test before a predetermined time.

38. The method of claim 1, wherein the alert signal is based on a determination that a result associated with a medical test is outside a predetermined range.

39. The method of claim 1, wherein the residence includes at least one of a home, a condominium, an apartment, an assisted living facility, and an independent living facility.

40. The method of claim 1, including:
causing a recommendation of at least one medical service and at least one non-medical service based on the result of the first needs assessment survey; and
causing a performance of the at least one medical service and the at least one non-medical service.

41. The method of claim 1, including causing downloading of software to a home controller, the software being structured to allow the new sensor to send data to the remote location via the home controller.

42. An apparatus for providing in-residence care to a person, the apparatus comprising:
a controller located at the residence of the person, the controller being structured to allow a plurality of medical and non-medical sensors to be connected to the controller;
a medical sensor located at the residence and operatively coupled to the controller;
a non-medical sensor located at the residence and operatively coupled to the controller;
a first audio/video teleconferencing system located at the residence;
a database server located outside the residence and in communication with the controller, the database server receiving data from the medical sensor and the non-medical sensor, the database server generating an alert based on the data; and
a second audio/video teleconferencing system located outside the residence, the second audio/video teleconferencing system communicating with the first audio/video teleconferencing system.

43. The apparatus of claim 42, wherein the controller comprises a plurality of controllers.

44. The apparatus of claim 42, wherein the medical sensor and the non-medical sensor are wirelessly coupled to the controller.

45. The apparatus of claim 42, wherein the database server is programmed to transmit a reminder to the residence, the reminder being to perform a medical procedure.

46. The apparatus of claim 45, wherein transmitting the reminder includes playing a prerecorded audio message over a telephone line.

47. The apparatus of claim 45, wherein transmitting the reminder includes playing a prerecorded audio/video message on the first audio/video teleconferencing system.

48. The apparatus of claim 42, wherein the database server is programmed to transmit a reminder to the residence, the reminder being to perform a non-medical procedure.

49. The apparatus of claim 48, wherein transmitting the reminder includes playing a prerecorded audio message over a telephone line.

50. The apparatus of claim 48, wherein transmitting the reminder includes playing a prerecorded audio/video message on the first audio/video teleconferencing system.

51. The apparatus of claim 42, wherein the database server is programmed to transmit a message to the residence in response to the alert.

52. The apparatus of claim 42, wherein the controller is operatively coupled to a wireless receiver to receive data from a visitor in the residence.

53. The apparatus of claim 42, wherein the database server is programmed to generate a report associated with a person living in the residence and automatically transmitting the report to a family member of the person.

54. The apparatus of claim 53, wherein a period and a type associated with the report are selectable by the family member via a global network.

55. The apparatus of claim 42, wherein the data from the medical sensor includes data from at least one of a weight scale, a vitals device, a blood pressure device, a blood sugar device, and a heart rate device.

56. The apparatus of claim 42, wherein the data from the non-medical sensor includes data from at least one of an alarm system, a motion detector, a door sensor, a window sensor, and a pressure sensor.

57. A remote station for providing in-residence care to a person, the apparatus comprising:
a first module for receiving data from a controller located at the residence of the person, the controller being connected a plurality of medical and non-medical sensors;
a second module for receiving audio/video signals from a first audio/video teleconferencing system located at the residence;
a database storing data received from the medical sensor and the non-medical sensor, the database generating an alert based on the data; and
a second audio/video teleconferencing system to communicate with the first audio/video teleconferencing system.
